(19) United States
(12) Patent Application Publication
(10) Pub. No.: US 2010/0278796 A1
(43) Pub. Date: Nov. 4, 2010

(54) Method for Improving the Digestibility and the Assimilability of Cereals and/or Fibres in a Monogastric Herbivorous Animal

(75) Inventor: Claudette Berger, Mennecy (FR)

Correspondence Address: MCANDREWS HELD & MALLOY, LTD, 500 WEST MADISON STREET, SUITE 3400, CHICAGO, IL, 60661, US

(73) Assignee: DANISCO A/S, Copenhagen (DK)

(21) Appl. No.: 12/765,220

(22) Filed: Apr. 22, 2010

(30) Foreign Application Priority Data
Apr. 30, 2009 (EP) 09305387.4
Mar. 23, 2010 (EP) 10157431.7

(51) Int. Cl. A61K 35/74; A23K 1/18; A23K 1/16; A61P 3/10; A61P 1/00

(52) U.S. Cl. 424/93.45; 426/2; 426/61; 424/93.46

(57) ABSTRACT

The invention relates to a method for improving the digestibility and assimilability of fibres and/or cereals in a monogastric herbivorous animal comprising the step of administering to said monogastric herbivorous animal an effective amount of at least one strain of bacterium selected fromselected from the group consisting of strains of bacteria of the genera Lactobacillus, Lactococcus, Propionibacterium, Bifidobacterium and Bacillus. The invention also relates to a feed supplement for a monogastric herbivorous animal comprising at least one strain of bacterium selected fromselected from the group consisting of strains of bacteria of the genera Lactobacillus, Lactococcus, Propionibacterium, Bifidobacterium and Bacillus.

### RELATED APPLICATIONS

The present application is filed as a U.S. application claiming the benefit of priority to European Patent Application No. 09305387.4, which was filed on Apr. 30, 2009, and European Patent Application No. 10157431.7, which was filed on Mar. 23, 2010. The entire text of the aforementioned applications is incorporated herein by reference in its entirety.

### FIELD OF THE INVENTION

The present invention relates to a method making it possible to improve the digestibility and assimilability of cereals and/or fibres in a monogastric herbivorous animal.

### BACKGROUND OF THE INVENTION

Various kinds of microorganisms in significant quantities colonize the gastro-intestinal tract and interact with each other in a series of complex relationships. In the ruminant herbivores, these microorganisms are essential in all the compartments of the gastro-intestinal tract and contribute in particular to the efficiency of the digestion of fodder and in particular in the destruction of plant cell walls and the digestion of fibres. However, the microorganisms naturally present in the gastro-intestinal tract of ruminants do not possess the battery of enzymes sufficient to achieve complete and optimum digestion of fibres. Thus, improving the digestibility of fibres in ruminants is the subject of comprehensive studies, giving rise to a great deal of hope. In fact, if microorganisms, or probiotics, with a better ability to decompose fibres or their components were discovered or developed by genetic engineering, these microorganisms could extract a greater proportion of the available nutrients from the fodder consumed, which would make it possible to significantly reduce the feed rations of the ruminants while retaining a similar energy intake.

U.S. Pat. No. 6,951,643 and U.S. Pat. No. 7,470,531 disclose the use of Propionibacterium P169 in polygastric herbivorous animals (ruminants) in order to increase the yield of protein and fats in milk. Moreover, U.S. Pat. No. 5,534,271 and U.S. Pat. No. 5,529,793 for their part disclose the combined action of a strain producing lactic acid and a strain consuming lactic acid for the production of milk and meat in ruminants. Finally U.S. Pat. No. 6,887,489 and U.S. Pat. No. 6,455,063 disclose the use of Propionibacterium P63 in combination with a lactic acid producing strain for reducing acidosis in bovines or reducing diarrhoea in pigs. However, none of these documents discloses the administration of microorganisms capable of improving the energy efficiency in a monogastric herbivorous animal which has a digestive mechanism totally different from that of ruminants. In fact, whereas the digestion of ruminants is essentially microbial and takes place in the rumen, the digestion of monogastric herbivores is essentially chemical and takes place primarily in the small intestine and the large intestine after the feed ration has passed through the stomach. Moreover, it is not possible to influence the microbial population of the large intestine of monogastric herbivores, as is done in the case of ruminants. In fact, unlike ruminants, in monogastric herbivores, the microorganisms must pass through the stomach and the small intestine before arriving at the sites where they can act. Thus, most of the microorganisms will be destroyed on contact with the acid pHs of the stomach. The administration of probiotics by oral route to monogastric herbivores is therefore linked to the latter's resistance to acid pHs. As a result, for these different reasons, it is not possible to foresee the behaviour of a probiotic normally used for ruminants in monogastric herbivores.

Moreover, few studies have been carried out on the administration of probiotic strains to monogastric herbivores.

The probiotic strain Lactobacillus pentosus WE7 which is a strain of equine origin, potentially endowed with in vitro properties, has been found to cause in young foals, apart from an increase in episodes of diarrhoea, abnormal clinical signs such as anorexia and depression (J. Scott Weese and Joyce Rousseau, JAVMA, Vol. 226, No. 12, Jun. 15, 2005).

Moreover, other strains of Lactobacillus of equine origin have been administered to young foals for the purpose of evaluating their efficacy in the treatment of clinically significant diarrhoea (Yuyama et al. Evaluation of a host-specific Lactobacillus probiotic in neonatal foals. J Appl Res Vet Med 2004; 2:26-33).

None of these publications discloses the effect of microorganisms on the improvement of the digestibility and assimilability of cereals and/or fibres in monogastric herbivores.

Recently, other studies have been carried out on horses, concerning the impact of a yeast, Saccharomyces cerevisiae, on the digestibility of fodder and particularly on the digestibility of fibres (Glade, M. J. (1991) Journal of Equine Veterinary Science 11(1): 10-16; Glade, M. J. (1991) Journal of Equine Veterinary Science 11(6): 323-329; Glade, M. J. (1992) Supplement to proceedings of Alltech's eighth annual symposium: 1-26; Glade, M. J. and L. M. Biesik (1986) Journal of Animal Science 62: 1635-1640; Glade, M. J. and M. D. Sist (1988) Nutrition Reports International 37: 11-17; Hall, R. R., S. G. Jackson, et al. (1990) Journal of Equine Veterinary Science 10(2): 130-134; Hausenblasz, J., J. Szuco, et al. (1993) 9^{th} Biotechnology in the Feed Industry symposium, Lexington, Ky., Alltech Technical Publications; Hill, J. and S. Gutsell (1998) BSAS annual meeting, Scarborough, UK, BSAS Publ; Kim, S. M., C. M. Kim, et al. (1991) Korean Journal of Animal Nutrition and feedstuff 15(5): 272-280; Medina, B. (2003) Dijon, France, University of Burgundy: 159; Moore, B. E. and K. E. Newman (1994) Journal of Animal Science 72(Suppl 1): 261; Pagan, J. D. (1990) Journal of Animal Science 68(Suppl. 1): 371).

Identifying other microorganisms capable of improving the digestibility and assimilability of cereals and/or fibres in a monogastric herbivorous animal therefore constitutes a major challenge to researchers and provides an unmet need in the industry.

### BRIEF SUMMARY OF THE INVENTION

In order to address an unmet need in the industry, the present invention provides methods of and compositions for improving the digestibility and assimilability of fibres and/or cereals in a monogastric herbivorous animal. In exemplary embodiments, the methods comprise administering to the monogastric herbivorous animal an effective amount of at least one strain of bacterium selected from selected from the group consisting of strains of bacteria of the genera Lactobacillus, Lactococcus, Propionibacterium, Bifidobacterium and Bacillus. The invention also relates to a feed supplement for a monogastric herbivorous animal comprising at least one strain of bacterium selected fromselected from the group consisting of strains of bacteria of the genera Lactobacillus, Lactococcus, Propionibacterium, Bifidobacterium and Bacillus.

In specific embodiments, step of administering to said monogastric herbivorous animal an effective amount of at least one strain of bacterium selected from the group consisting of strains of bacteria of the genera Lactobacillus, Lactococcus, Propionibacterium, Bifidobacterium and Bacillus consists of administering an effective amount of a mixture of at least two strains of bacteria selected from the group consisting of strains of bacteria of the genera Lactobacillus, Lactococcus, Propionibacterium, Bifidobacterium and Bacillus. More particularly, the mixture of strains of bacteria comprises at least one strain of bacterium of the genus Lactobacillus.

In preferred embodiments the method is carried out using strains of bacteria selected from strains of bacteria of the species L. paracasei, L. casei, L. acidophilus, L. buchnerii, L. farciminis, L. rhamnosus, L. reuteri, L. fermentum, L. brevis, L. lactis and L. plantarum; Lactococcus cremoris and Lactococcus lactis; Propionibacterium jensenii, Propionibacterium acidipropionici, Propionibacterium freudenreichii and Propionibacterium freudenreichii ssp shermanii; Bifidobacterium longum, Bifidobacterium lactis and Bifidobacterium animalis; Bacillus licheniformis, Bacillus subtilis and Bacillus cereus. For example, it is contemplated that the mixture of at least two strains of bacteria is a mixture of at least one strain of L. plantarum and at least one strain of Propionibacterium jensenii. In more particular examples, the mixture is a mixture of L. plantarum Lp115 and Propionibacterium jensenii P63.

In particular embodiments, the strains of bacteria are preferably inactivated.

The methods of the invention may be carried out such that the effective amount of at least one strain of bacterium is administered to said monogastric herbivorous animal by supplementing food intended for said animal with said effective amount of at least one strain of bacterium.

The animal may be any monogastric herbivorous animal. In preferred embodiments, the monogastric herbivorous animal is selected from the members of the Equidae and Suidae.

In exemplary embodiments, the monogastric herbivorous animal is a member of the horse family. More specifically, the monogastric herbivorous animal is a horse or a pony.

Another aspect of the invention contemplates a feed supplement for a monogastric herbivorous animal comprising at least one strain of bacterium selected from the group consisting of strains of bacteria of the genera Lactobacillus, Lactococcus, Propionibacterium, Bifidobacterium and Bacillus. In exemplary embodiments, the feed supplement comprises at least one strain of bacterium selected from the group consisting of the species L. paracasei, L. casei, L. acidophilus, L. buchnerii, L. farciminis, L. rhamnosus, L. reuteri, L. fermentum, L. brevis, L. lactis and L. plantarum; Lactococcus cremoris and Lactococcus lactis; Propionibacterium jensenii, Propionibacterium acidipropionici, Propionibacterium freudenreichii and Propionibacterium freudenreichii ssp shermanii; Bifidobacterium longum, Bifidobacterium lactis and Bifidobacterium animalis; Bacillus licheniformis, Bacillus subtilis and Bacillus cereus. In more specific examples, the feed supplement comprises at least one strain of bacterium selected from L. plantarum Lp115 and Propionibacterium jensenii P63.

Also contemplated by the present invention is an improved feed wherein the feed comprises a feed supplement for a monogastric herbivorous animal comprising at least one strain of bacterium selected from the group consisting of strains of bacteria of the genera Lactobacillus, Lactococcus, Propionibacterium, Bifidobacterium and Bacillus.

The present invention further contemplates a method of regulating glycaemia in a monogastric herbivorous animal, said method comprising the step of administering to said animal a feed supplement comprising at least one strain of bacterium selected from the group consisting of strains of bacteria of the genera Lactobacillus, Lactococcus, Propionibacterium, Bifidobacterium and Bacillus.

Another aspect of the invention relates to a method of reducing digestive disturbances induced by intense physical exercise and/or stress in a monogastric herbivorous animal, said method comprising the step of administering to said animal at least one strain of bacterium selected from the group consisting of strains of bacteria of the genera Lactobacillus, Lactococcus, Propionibacterium, Bifidobacterium and Bacillus.

### BRIEF DESCRIPTION OF SEVERAL VIEWS OF THE DRAWINGS

FIG. 1: the FIGURE represents postprandial development of the concentration of blood glucose measured in the horses receiving the treatments TSH-MS01, MS02 and MS03 (n=6).

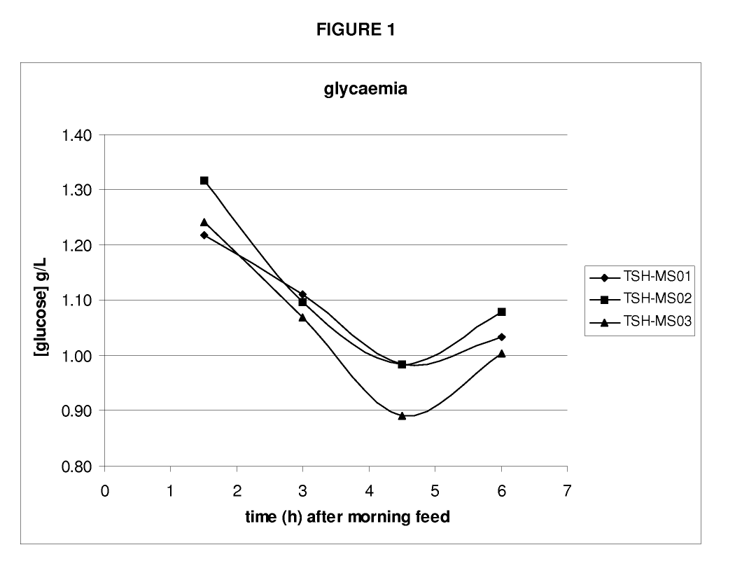

### DETAILED DESCRIPTION OF THE INVENTION

The present invention relates to a method for improving the digestibility and assimilability of cereals and/or fibres in a monogastric herbivorous animal. In fact, surprisingly and unexpectedly, the inventors have shown that certain bacteria possess the property of promoting the digestion of cereals and/or fibres from the feed ration in monogastric herbivorous animals. These bacteria belong to the genera Lactobacillus, Lactococcus, Propionibacterium, Bifidobacterium and Bacillus. The invention therefore relates to a method for improving the digestibility and assimilability of cereals and/or fibres in a monogastric herbivorous animal comprising the step of administering to said monogastric herbivorous animal an effective amount of at least one strain of bacterium selected from the group consisting of strains of bacteria of the genera Lactobacillus, Lactococcus, Propionibacterium, Bifidobacterium and Bacillus.

Within the meaning of the invention, “digestibility” is a criterion which defines the degree to which organic matter can be digested by an animal. In the plant kingdom (fodder for example) this criterion generally decreases as the level of lignin in a plant increases. The vegetative parts of plants have much higher digestibilities than the stalks for example.

By “assimilability”, is meant, within the meaning of the invention, the ability of a food to be catabolized to nutrients which can be assimilated by the blood.

Within the meaning of the invention, by “fibres”, is meant a natural plant substance constituted mainly by a carbohydrate polymer. Non-limitative examples of fibres are the celluloses, hemicelluloses, pectins, proteoglycans, etc. Non-limitative examples of sources of fibres for herbivorous animals are grass, hay, alfalfa, straw, grains, etc., but also other vegetables, such as carrots.

Within the meaning of the invention, by “cereal” is meant any plant cultivated for its grains which can be used in animal feed. The grains of these plants are also specifically meant. Non-limitative examples of cereals according to the invention are maize, rice, wheat, barley, oats, rye, millet, buckwheat, quinoa and sesame.

More generally, by “digestibility and assimilability of fibres and/or cereals”, is meant the degree to which fibres or cereals can be digested by the animal to soluble carbohydrates, fatty acids and amino acids which can be assimilated by the animal. An increase in the digestibility and assimilability of fibres and/or cereals results in an increase in the proportion of available nutrients extracted from the fodder or cereals consumed.

By “monogastric herbivorous animal”, is meant any animal the diet of which is constituted mainly by plant matter and which possesses only one stomach compartment. Examples of monogastric herbivorous animals are the members of the horse family and pigs.

By “administer”, is meant the action of introducing at least one strain of bacterium according to the invention into the animal's gastro-intestinal tract. More particularly, this administration is an administration by oral route. This administration can in particular be carried out by supplementing the feed ration intended for the animal with said at least one strain of bacterium, the thus supplemented feed ration then being ingested by the animal. The administration can also be carried out using a stomach tube or any other means making it possible to directly introduce said at least one strain of bacterium into the animal's gastro-intestinal tract.

By “effective amount”, is meant a quantity of bacteria sufficient to allow improvement of the digestibility of the fibres. This effective amount can be administered to said monogastric herbivorous animal in one or more doses.

By “at least one strain”, is meant a single strain but also mixtures of strains comprising at least two strains of bacteria.

By “a mixture of at least two strains”, is meant a mixture of two, three, four, five, six or even more strains.

In a first aspect the invention therefore relates to a method for improving the digestibility and assimilability of fibres and/or cereals in a monogastric herbivorous animal comprising the step of administering to said monogastric herbivorous animal an effective amount of at least one strain of bacterium selected from the group consisting of strains of bacteria of the genera Lactobacillus, Lactococcus, Propionibacterium, Bifidobacterium and Bacillus.

This method allows the monogastric herbivorous animal to derive greater benefit in terms of energy from feed based on fibres and cereals, and as a result, starting from the same calorie intake, to increase the energy available to its metabolism. This is advantageous for the livestock farmer who can thus optimize the cost of the feed rations. In fact, he can either reduce the animal's feed rations for the same energy intake or reduce the quantity of starchy cereals and replace it with less expensive fibre-rich fodder, which allows him to make a financial saving.

This method also allows the animal to derive greater benefit from the energy intake from the starch present in the cereals, which allows it to have more energy which can be mobilized during effort (strength or speed). This is advantageous for example for race horses or sport horses.

This method also allows the animal to benefit from the energy intake from the fibres present in the fodder, which allows it to have energy which can be mobilized during prolonged effort (endurance). This is advantageous for example for touring and recreation horses.

Finally, this method makes it possible to increase the energy available to the monogastric herbivorous animal while limiting fluctuations in its glycaemia. This has the consequence of prolonging and stabilizing the animal's energy intake in the long term.

Moreover, whereas it has been observed that intense physical exercise, for example during sporting events (races, competitions etc.) and conditions of stress (for example during transport, a change of location, change of environment, change of staff, of competition etc.) result in digestive disturbances in animals (see in particular Goachet et al., Comparative Exercise Physiology 5(3-4); 143-151), the method according to the invention makes it possible to correct these disturbances. The animals to which at least one strain of bacterium according to the invention is administered display improved digestibility and assimilability of fibres and cereals, even under conditions of physical effort and stress. The strains of bacteria according to the invention have the effect of preventing digestive disorders.

The invention thus relates to a method for reducing digestive disturbances induced by intense physical exercise or stress in a monogastric herbivorous animal comprising the step of administering to said animal an effective amount of at least one strain of bacterium selected from the group consisting of strains of bacteria of the genera Lactobacillus, Lactococcus, Propionibacterium, Bifidobacterium and Bacillus.

Typically, in order to have a preventative and recuperative effect, the strains of bacteria according to the invention can be administered daily over a period starting 3 months, 3 weeks, 2 weeks, 1 week or 1 day before the intense physical exercise and/or the stressful event and finishing 2 months, 2 weeks or 1 week after the intense physical exercise and/or the stressful event.

In a particular embodiment, the methods according to the invention are characterized in that the step of administering to said monogastric herbivorous animal an effective amount of at least one strain of bacterium selected from the group consisting of strains of bacteria of the genera Lactobacillus, Lactococcus, Propionibacterium, Bifidobacterium and Bacillus consists of administering an effective amount of a mixture of at least two strains of bacteria selected from the group consisting of strains of bacteria of the genera Lactobacillus, Lactococcus, Propionibacterium, Bifidobacterium and Bacillus.

In a particular manner, the mixture of strains of bacteria according to the invention comprises at least one strain of bacterium of the genus Lactobacillus. Examples of mixtures of strains of bacteria according to the invention are in particular a mixture comprising at least two strains of the genus Lactobacillus, or a mixture comprising at least one strain of the genus Lactobacillus and at least one strain of the genus Propionibacterium.

The proportions can vary from 1% to 99%, more advantageously from 25% to 75% and even more advantageously approximately 50% for each strain. In a mixture comprising more than two strains, the strains are preferentially present in substantially equal proportions in the mixture.

In yet another embodiment of the invention, the strains of bacteria of the genus Lactobacillus are not bacteria of the strain Lactobacillus pentosus WE7.

According to an embodiment of the invention, the strains of the genus Lactobacillus are in particular selected from the species L. paracasei, L. casei, L. acidophilus, L. buchnerii, L. farciminis, L. rhamnosus, L. reuteri, L. brevis, L. fermentum, L. lactis and L. plantarum.

More particularly, a strain of the species L. plantarum is the strain L. plantarum Lp115, deposited under the Budapest Treaty on 9 Feb. 2009, in the Deutsche Sammlung von Mikroorganismen and Zellkulturen GmbH (DSMZ, Inhoffenstr. 7 B, D-38124 Braunschweig, Germany) under number DSM22266 by Danisco Deutschland GmbH (Bush-Johannsen-Str. 1, 25899 Niebüll, Germany).

The strains of the genus Lactococcus are in particular selected from the species Lactococcus cremoris and Lactococcus lactis.

The strains of the genus Propionibacterium are in particular selected from strains of the species Propionibacterium jensenii, Propionibacterium acidipropionici, Propionibacterium freudenreichii and Propionibacterium freudenreichii ssp shermanii. A particular strain of the species Propionibacterium jensenii according to the invention is the strain Propionibacterium jensenii P63, deposited under the Budapest Treaty on 15 Jan. 2009, in the Deutsche Sammlung von Mikroorganismen and Zellkulturen GmbH (DSMZ, Inhoffenstr. 7 B, D-38124 Braunschweig, Germany) under number DSM22192 by Danisco Deutschland GmbH (Bush-Johannsen-Str. 1, 25899 Niebüll, Germany).

The strains of the genus Bifidobacterium are in particular selected from strains of the species Bifidobacterium longum, Bifidobacterium lactis and Bifidobacterium animalis.

The strains of the genus Bacillus are in particular selected from strains of the species Bacillus licheniformis, Bacillus subtilis and Bacillus cereus.

In an embodiment of the invention, the mixture of at least two strains of bacteria is a mixture of at least one strain of the species L. farciminis and at least one strain of the species L. rhamnosus.

The mixtures of strains of the genus Lactobacillus, and more particularly the mixture of at least one strain of the species L. farciminis and at least one strain of the species L. rhamnosus, are particularly suitable for monogastric herbivores having to exert a great effort over a short period. In fact, as evidenced by the experimental results of the invention, these particular mixtures of strains make it possible to reproduce the effect of a starch-rich energy diet, although the ration is rich in fibres. As a result, these mixtures allow the animal to derive greater benefit from its feed ration, rapidly after absorption of its ration. This type of mixture is therefore particularly suitable for race horses or sport horses.

In another embodiment of the invention the mixture of at least two strains of bacteria is a mixture of at least one strain of L. plantarum and at least one strain of Propionibacterium jensenii. In particular, the mixture of at least two strains of bacteria is a mixture of Propionibacterium jensenii P63 and of L. plantarum Lp115. In a particular embodiment, the strains of Propionibacterium jensenii (in particular P63) and of L. plantarum (in particular Lp115) are present in the mixture in substantially equal quantities (approximately 50%/50%). Such a mixture is in particular suitable for administration to a monogastric herbivore which has a low-starch, fibre-rich diet. In fact, as described in the experimental part, this particular mixture of bacteria allows the animal to derive greater benefit from the plant fibres, over a longer period. This mixture is therefore particularly suitable for animals having an endurance activity, such as in particular touring and recreation horses.

According to a particular embodiment, the methods according to the invention also comprise the step of administering other microorganisms, said microorganisms being selected from the group comprising in particular the lactic bacteria, probiotic microorganisms, yeasts and fungi (for example Penicillium and Geotrichum).

According to an embodiment of the invention, the strains of bacteria are inactivated before their administration to the herbivorous animal. The inactivation makes it possible to significantly reduce the microorganisms' ability to reproduce without significantly affecting their enzymatic activity. Typically, following the inactivation process, the number of microorganisms capable of reproducing is reduced by a factor greater than X, X being selected from the following values: 10⁴, 10⁵, 10⁶, 10⁷, 10⁸, 10⁹, 10¹⁰ and 10¹¹.

Typically, the microorganisms can be inactivated by a heat shock treatment. For example, the microorganisms can be exposed to temperatures comprised between 40° C. and 70° C. The duration of the heat shock treatment will depend on the chosen temperature and the microorganism to be inactivated. For example, the inactivation method can be carried out over a period of time comprised between 15 minutes and 96 hours. For example also, the microorganisms can be exposed to temperatures comprised between 60° C. and 70° C. for a period of time comprised between 20 and 40 hours.

Other techniques can be used to inactivate the microorganisms, such as for example ionization or photoinactivation (inactivation by light). The microorganisms can also be inactivated by keeping them for long periods at a temperature or humidity level which is not compatible with their viability.

The inactivation of the strains of bacteria according to the invention has the consequence of preventing the multiplication and development of the bacteria while preserving their enzyme battery and therefore their fibre-digestibility properties. Moreover, the inactivation of the strains means that the strains will not enter into competition with the fibrolytic, cellulolytic and amylolytic intestinal flora, while releasing their enzyme content into the medium.

According to the invention, the digestibility of the fibres is considered “improved” if the fibres are better digested by the animal in the presence of said at least one strain of bacterium. In a non-limitative manner, methods which can be used to measure the digestibility of the fibres are the methods of measuring the final fermentation products. For instance, measurement of lactic acid, for example by an enzymatic colorimetric method, and measurement of volatile fatty acids (VFAs), for example by gas chromatography as described by Jouany JP and Senaud J in Reprod Nutr Dev. 1982; 22(5):735-52, are suitable. Thus, using these methods, a person skilled in the art is able to compare digestibility in the presence and in the absence of the strains of bacteria according to the invention.

Typically, said at least one strain according to the invention is administered to said monogastric herbivorous animal during and/or outside feed intakes.

In a particular embodiment of the invention, said effective amount of at least one strain of bacterium is administered to said monogastric herbivorous animal by supplementing a feed intended for said animal with said effective amount of at least one strain of bacterium. By “supplementing”, within the meaning of the invention, is meant the action of incorporating the effective amount of bacteria according to the invention directly into the feed intended for the animal. Thus, the animal, when feeding, ingests the bacteria according to the invention which can then act to increase the digestibility and assimilability of the fibres and/or cereals contained in the animal's feed.

Thus, another subject of the invention relates to a feed supplement for a monogastric herbivorous animal comprising at least one strain of bacterium selected from the group consisting of strains of bacteria of the genera Lactobacillus, Lactococcus, Propionibacterium, Bifidobacterium and Bacillus.

In an embodiment of the invention, the strains of bacteria of the genus Lactobacillus are not selected from strains of the species Lactobacillus pentosus.

In yet another embodiment of the invention, the strains of bacteria of the genus Lactobacillus are not bacteria of the strain Lactobacillus pentosus WE7.

Typically, the strains of the genus Lactobacillus are in particular strains of the species L. paracasei, L. casei, L. acidophilus, L. buchnerii, L. farciminis, L. rhamnosus, L. reuteri, L. fermentum, L. brevis, L. lactis and L. plantarum. More particularly, a strain of the species L. plantarum is the strain L. plantarum Lp115, deposited under the Budapest Treaty on 9 Feb. 2009, in the Deutsche Sammlung von Mikroorganismen und Zellkulturen GmbH (DSMZ, Inhoffenstr. 7 B, D-38124 Braunschweig, Germany) under number DSM22266 by Danisco Deutschland GmbH (Bush-Johannsen-Str. 1, 25899 Niebüll, Germany).

The strains of the genus Lactococcus are in particular selected from strains of the species Lactococcus cremoris and Lactococcus lactis.

The strains of the genus Propionibacterium are in particular selected from strains of the species Propionibacterium jensenii, Propionibacterium acidipropionici, Propionibacterium freudenreichii and Propionibacterium freudenreichii ssp shermanii. A particular strain of the species Propionibacterium jensenii is the strain Propionibacterium jensenii P63, deposited under the Budapest Treaty on 15 Jan. 2009, in the Deutsche Sammlung von Mikroorganismen und Zellkulturen GmbH (DSMZ, Inhoffenstr. 7 B, D-38124 Braunschweig, Germany) under number DSM22192 by Danisco Deutschland GmbH (Bush-Johannsen-Str. 1, 25899 Niebüll, Germany).

The strains of the genus Bifidobacterium are in particular selected from strains of the species Bifidobacterium longum, Bifidobacterium lactis and Bifidobacterium animalis.

The strains of the genus Bacillus are in particular selected from strains of the species Bacillus licheniformis, Bacillus subtilis and Bacillus cereus.

According to the invention, by “at least one strain” is meant one or more strains of bacteria. By “several strains” is meant a mixture of at least two strains of bacteria. In an embodiment of the invention, said mixture is a mixture of at least one strain of the species L. farciminis and at least one strain of the species L. rhamnosus. In another embodiment of the invention, said mixture is a mixture of Propionibacterium jensenii and L. plantarum. More particularly, said mixture is a mixture of Propionibacterium jensenii P63 and L. plantarum Lp115.

The invention also relates to a method for regulating the glycaemia in a monogastric herbivorous animal, said method comprising the step of administering to said animal a feed supplement comprising at least one strain of bacterium according to the invention selected from L. plantarum Lp115 and Propionibacterium jensenii P63. Actually, these particular strains of bacteria have the property of limiting variations in glycaemia after intake of the feed ration: the glycaemia is maintained at a lower level, and its fluctuations are limited. This regulation of glycaemia provides evidence of the diffusion of the animal's energy intake in a regular and prolonged manner over time (cf. experimental part, §2.1).

As described previously, and according to a particular embodiment, said strains of bacteria in said supplements according to the invention are inactivated.

According to an embodiment of the invention, said feed supplement for a monogastric herbivorous animal comprising at least one strain of bacterium also comprises other microorganisms, said microorganisms being selected from the group comprising in particular the lactic bacteria, probiotic microorganisms, yeasts and fungi (for example Penicillium and Geotrichum).

Another subject of the invention relates to a feed for a monogastric herbivorous animal, characterized in that it is supplemented with a feed supplement as described previously.

According to the invention, the feed for a monogastric herbivorous animal which can be supplemented with said feed supplement is typically selected from feeds containing fibres and/or cereals. Examples of feeds according to the invention are grass, hay, alfalfa, straw, grains, or any other type of fodder used for feeding herbivores, but also any type of granulated feed, in particular based on wheat bran, oat husks, alfalfa, barley, maize, fruit pomace, cane molasses, low-grade rice flour, straw, soya oil cake, etc.; or also other types of vegetables, such as in particular carrots.

Typically, the feed is supplemented with said feed supplement so that the animal receives an effective amount of bacteria to improve the digestibility and assimilability of the fibres and/or cereals contained in the animal's feed.

According to the present invention, said effective amount of said at least one strain of bacterium is typically comprised between 10⁵ CFU and 10¹³ CFU per animal and per day, particularly between 10⁷ CFU and 10¹² CFU per animal and per day, more particularly between 10⁸ CFU and 10¹¹ CFU per animal and per day, even more particularly approximately 10¹⁰ CFU per animal and per day. When the bacteria are inactivated, the quantities described previously are calculated before inactivation.

In a particular manner, within the meaning of the invention, said monogastric herbivorous animal is selected from the Equidae and Suidae. More particularly the monogastric herbivore is a member of the horse family, typically selected from the group consisting of horses, ponies and donkeys. In a particular manner, the animal which is the subject of the method according to the invention is the horse or pony.

Other aspects and advantages of the present invention are described in the FIGURE and the following examples, which must be considered as illustrative and not limiting the scope of the invention.

### EXAMPLES

### Glossary

ADF Acid Detergent Fibre
ADL Acid Detergent Lignin
dX digestibility of constituent X
RE Raw energy
g gram
kg kilogram
RM Raw material
OM Organic matter
DM Dry matter
IDM Ingested dry matter
NDF Neutral Detergent Fibre
NS Not significant
BW Body weight
R² Correlation coefficient
SD Standard deviation

### Example 1

### 1. Material and Methods

The experiments described below were carried out on the experimental platform of AgroSup Dijon, France (previously ENESAD Dijon).

1.1. Management and Feeding of the Experimental Animals

1.1.1. Characteristics and Management of the Horses

Six French trotter geldings (age comprised between 3 and 9 years) of initial body weight comprised between 423 kg and 512 kg and initial body condition score comprised between 1 and 2 established according to the criteria of the national stud farms (Haras Nationaux), were used in this test. They were placed in individual boxes (12.5 m²), on a bedding of wood shavings (Tier Wohl®, Rettenmaier). This type of litter made it possible to avoid the uncontrolled ingestion of plant fibres. They had a block of mineral salts which they could access at will (Dolmin Salt, Lactona) and had free access to an automatic drinking trough.

The horses were up to date with their worming (Equimax, Virbac) and vaccination (Equillis Prequenza TE, Intervet and Proteqflu TE, Merial).

1.1.2. Experimental Diets

a. Basic Diet and Distribution Modalities

The animals received a granulated feed complemented with a meadow hay (Tables 1 and 2). The hay and granulated feed were kept in a proportion of 60:40 (% of IDM). The quantity of hay and granulated feed distributed daily was 1.5 kg of RM (Raw Material) and 1 kg of RM respectively per 100 kg of BW. The ration was divided into four portions: given at 8:30 and 17:00 in the case of the granules; at 10:00 and 16:00 in the case of the hay. The horses' daily ration made it possible to cover 120% of the energy needs in the case of maintenance and very light work of the animals based on the INRA recommendations (1990).

TABLE 1
Composition of the granulated feed DP Puissance (Evialis, France)
Raw materials

Wheat bran (40%)
Oat husks (10%)
Oats (10%)
Alfalfa (10%)
Barley (7%)
Fruit pomace (7%)
Cane molasses (6%)
Low-grade rice flour
Straw
Soya oil cake
Calcium carbonate
Salt
Sodium bicarbonate

TABLE 2
Composition of the feeds in the basic ration

| | | Granulated |
|---|---|---|
| | Hay | feed |
| Dry matter (DM), % RM | 88.95 | 89.1 |
| Organic matter (OM), % DM | 91.9 | 91.7 |
| Neutral Detergent Fibre (NDF), | 64.6 | 42.0 |
| % DM | | |
| Acid Detergent Fibre (ADF), % DM | 35.9 | 20.7 |
| Acid Detergent Lignin (ADL), % DM | 4.6 | 6.0 |
| Horse feed unit (HFU/kg DM) | 0.43 | 0.90 |
| Horse digestible crude protein | 33.7 | 104.3 |
| (HDCP/kg DM) | | |

b. Supplementation with Products TSH-MS01, 02, 03:

The three tested products are the following:
TSH-MS01: 50/50 Mixture of Lactobacillus plantarum Lp115 and Propionibacterium jensenii P63.
TSH-MS02: Placebo
TSH-MS03: Combination of two inactivated strains of Lactobacillus (mixture of L. farciminis and L. rhamnosus).

The three products to be tested were mixed beforehand with a soya oil cake and maize flour before being distributed to the animals with the morning feed of granules. The flour samples are denoted TSH-F1, TSH-F2 and TSH-F3. The combinations were made up as follows:
TSH-MS01 mixed with TSH-F1
TSH-MS02 mixed with TSH-F2
TSH-MS03 mixed with TSH-F3

The doses of product were prepared in individual two-gram sachets in the case of the TSHMS, stored at 4° C. and 50-gram sachets in the case of the TSH-F, stored at ambient temperature (<or =20° C.). The products TSH-MS01 and TSH-MS03 were used to supplement TSH-F1 and TSH-F3 at a level of 10¹⁰ CFU per horse and per day.

1.1.3. Schedule of Experiments

The test was carried out according to a latin square procedure 3×3 (3 periods×3 treatments). In this scenario, the two products TSH-MS01 and TSH-MS03 were tested against a placebo over 3 experimental periods with two animals per treatment (Table 3).

A twenty-day phase of adaptation to the diet was followed by three successive periods. Each period (approximately 42 days) was composed of a 21-day supplementation phase during which the products TSH-MS01 TSH-MS02 and TSH-MS03 were distributed and the physiological parameters measured, then a washout phase (no supplementation) for a minimum duration of 20 days.

Three groups of horses were constituted such that they were fairly homogeneous in terms of the weight of the horses. The three groups of animals received each of the three treatments in turn.

TABLE 3
Description of the schedule of experiments

| | Adaptation | 1^{st} period | 2^{nd} period | 3^{rd} period |
|---|---|---|---|---|
| | (20 days) | (42 days) | (42 days) | (42 days) |
| Group 1 | Basic diet | Diet | Diet | Diet |
| C1 and | | supplemented | supplemented | supplemented |
| C2 | | with | with | with |
| | | TSH-MS01 | TSH-MS02 | TSH-MS03 |
| Group 2 | Basic diet | Diet | Diet | Diet |
| C3 and | | supplemented | supplemented | supplemented |
| C4 | | with | with | with |
| | | TSH-MS02 | TSH-MS03 | TSH-MS01 |
| Group 3 | Basic diet | Diet | Diet | Diet |
| C5 and | | supplemented | supplemented | supplemented |
| C6 | | with | with | with |
| | | TSH-MS03 | TSH-MS01 | TSH-MS02 |

1.2. Study of the metabolic blood profile

1.2.1. Collection and preparation of the samples

For each supplementation phase, four blood samples were collected during the first six hours after the morning feed during each supplementation phase: 1½ hours, 3 hours, 4½ hours and 6 hours in order to determine the blood glucose concentrations. The samples were collected from the jugular vein in tubes pre-treated with K₂-oxalate calcium fluoride.

After each sample was collected, the plasma was obtained by centrifugation (4500 rpm, 5 min) then transferred to a new dry tube before being frozen at −20° C.

1.2.2. Analyses

The blood concentrations were determined using enzymatic kits from Kit Biomerieux (kit No. 61269).

1.3. Study of the Faecal Microflora and its Activity

1.3.1. Collection and Preparation of the Samples

For each experimental period, the samples of faeces were collected from the 6 horses 3 hours after the morning feed by rectal sampling.

A portion (10 g) was maintained at 38° C. under conditions of anaerobiosis up to the time of seeding. The other portion was filtered on nylon fabric (porosity 100 μm). The pH was immediately measured on the filtrate obtained. 1 ml aliquots of filtered sample were collected then frozen in microtubes for subsequent analysis of the concentrations of volatile fatty acids (VFAs) and lactate. For the assay of the VFAs, the samples were stabilized beforehand with a solution containing 100 μl of HgCl₂.

1.3.2. pH Measurements

The pH was measured using a soil pH meter (WTW, 340i, Germany) and suitable electrodes.

1.3.3. Assay of the Final Fermentation Products (VFA, Lactate)

The assay of the lactic acid was carried out according to a colorimetric enzymatic method (kit Enzy plus, Diffchamb™, SE 42131 Västra Frölunda, Sweden). The colorimetric assay was carried out by absorption spectrophotometry at λ=540 nm using a microplate reader (MRX revelation, Dynatech Laboratories, Guyancourt, France).

The VFAs were assayed by gas chromatography (Gas chromatograph model 437 A, United Technologies Packard, Zurich, Switzerland) according to the method described by Jouany (1982).

1.3.4. Microbial Counts

The counting of different groups of bacteria present in the faecal microbial ecosystem was carried out according to the standard (Hungate, 1969), modified (Grubb and Dehority, 1976) culture techniques and culture techniques adapted to horses (Baruc et al., 1983; Julliand et al., 1999). The counts expressed in CFUs were converted to log₁₀ CFU.

a. Preparation of the Inocula

The inocula were prepared by successive decimal dilutions of the faecal samples in a dilution medium under strict anaerobiosis (Bryant and Burkey, 1953). For each sample, three successive dilutions were seeded, in triplicate, in the different culture media.

b. Culture Media and Counts

The total anaerobic flora count was carried out according to the “roll tubes” technique (Hungate, 1969) on complete agar medium (Leeddle and Hespell, 1980) under strict anaerobiosis. The total anaerobic bacteria concentration was determined after incubation at 38° C. for 48 hours from the three repetitions carried out at the dilutions 10⁻⁶, 10⁻⁷, 10⁻⁸.

The cellulolytic flora count was carried out under strict anaerobiosis in tubes containing a strip of Whatman No. 1 paper as single cellulolytic substrate in 4 ml of medium (Halliwell and Bryant, 1963). The tubes were seeded with 1 ml of inoculum at 10⁻⁴, 10⁻⁵, 10⁻⁶. After incubation for 15 days at 38° C., the most probable number (MPN) of cellulolytic bacteria was estimated according to McGrady's method.

The amylolytic flora count was carried out in Petri dishes by deep seeding in suitable agar (thesis of M. Varloud, 2006). The count was carried out after incubation for 48 hours at 38° C. starting from the three repetitions of the dilutions (10⁻⁴, 10⁻⁵, 10⁻⁶).

The count of the lactate-using flora was carried out under strict anaerobiosis according to the “roll-tubes” method on selective medium (Mackie and Wi1kins, 1979). The bacteria concentration was determined after incubation at 38° C. for 48 hours for each dilution (10⁻⁴, 10⁻⁵, 10⁻⁶).

1.4. Measurement of the Apparent Total Digestibility of the Constituents of the Ration

1.4.1 Methodology

The apparent total digestibility (dX) of the constituent X was measured by total collection of faeces in digestibility harnesses over 5 days.

It was calculated using ADL (Acid Detergent Lignin) as undigested internal marker according to the equation (Miraglia et al., 1999):

$$dX = \frac{[ ( I \times Xi ) / ( I \times \% ADLi ) - ( F \times Xf ) / ( F \times \% AdLf ) ]}{[ ( I \times Xi ) / ( I \times \% ADLi ) ]}$$

with:

Xi: the X content of the ingested dry matter I;

Xf: the X content of the excreted dry matter F in the faeces;

% ADLi: ADL content of the ingested dry matter I;

% ADLf: ADL content of the excreted dry matter F;

and in the case of a diet composed of fodder/granules:

I×Xi=I_{fodder}×X_{fodder}+I_{granules}×X_{granules}

and

I×% ADLi=I_{fodder}×% ADL_{fodder}+I_{granules}×% ADL_{granules}

The total quantity of excreted faeces was weighed each day. Any uneaten feed was also weighed in order to determine the exact daily quantity of ingested DM.

Samples of faeces, uneaten feed and distributed feeds were collected each day (Table 4) and placed in a forced air oven at 65° C. until a constant weight was obtained in order to determine the DM. Once dry, the samples of faeces and waste collected during the 5 days of digestibility testing were ground (0.8 mm grid) and collected (pooled) for each animal. The samples of granulated feeds and hay were also ground and totalled at the end of the period.

TABLE 4
Sampling grid for distributed and wasted feeds and
droppings during the in vivo digestibility tests.

| | | |
|---|---|---|
| | | Quantity sampled in |
| | Total quantity | order to determine |
| Type of sample | collected/day | the DM |

| | | |
|---|---|---|
| Granulated feed | 500 | g |
| Hay | 1 | kg |

| | | |
|---|---|---|
| Faeces | Less than 10 kg | 10% |
| | More than 10 kg | 5% |
| Uneaten (hay and | Less than 600 g | 100% |
| granules) | From 600 g to 1500 g | 50% |
| | From 1500 g to 3000 g | 25% |
| | More than 3000 g | 5% |

1.4.2. Analyses:

The following analyses were carried out on each reconstituted sample:
determination of the dry matter, in an oven at 65° C., until a constant weight is reached;
assay of the parietal fractions (NDF, ADF, ADL) by the Van Soest method (Van Soest and Wine, 1967) at semi-automatic fodder analysis stations (Fibertech system M 1020 Extractor, Tecator, Hoganas, Sweden). The hemicelluloses, cellulose and all of the potentially digestible walls were then estimated from the differences between NDF-ADF, ADF-ADL and NDF-ADL respectively.
determination of the organic matter after passing through the oven (550° C. over 5 hours).

1.5 Statistical Analyses

The software used for the statistical processing of all the data was the SAS software version 6.12 (SAS/STAT, 1998).

1.5.1. Data on Digestibility, Microbial Counts and Fermentative Activity (pH, VFA, Lactic Acid):

The data were processed by variance analysis using the GLM (Generalized Linear Model) procedure. The adjusted means (Least Square means) were used to compare the differences between the treatments (pdiff function).

1.5.2. Blood Concentration (Glycaemia) Data:

The blood concentration data were processed by variance analysis (GLM procedure) with repeated measurements (SAS software repeated option) using the following general linear model. The adjusted means (Least Square means) were used to compare the differences between the treatments (pdiff function).

1.5.3. Statistical Levels

The statistical levels adopted for the variance analyses were as follows:

p<0.1: the results display a trend; p<0.05: the results are significantly different; p<0.01: the difference is very significant; p<0.001: the difference is highly significant.

2.2—Results and Discussions

2.1. Metabolism: Glycaemia

TABLE 5
Effect of the products TSH-MS01, 02 and 03 on the changes in the
measured glucose concentration in the horses' blood (n = 6)

| | | | Treatment | | | |
|---|---|---|---|---|---|---|
| Glycaemia | | | TSH- | TSH- | | |
| g/L | SD | R² | MS01 | MS02 | TSH-MS03 | Effects (1) |
| T_{+1.5} | 0.11 | 0.88 | 1.218 | 1.317 | 1.242 | H*** |
| T₊₃ | 0.10 | 0.86 | 1.110 | 1.097 | 1.070 | (p < 0.001) |
| T_{+4.5} | 0.16 | 0.83 | 0.983 | 0.983 | 0.892 | C** |
| T₊₆ | 0.06 | 0.88 | 1.033 | 1.078 | 1.003 | (p = 0.003) |

(1) significance level:
*p < 0.05;
**p < 0.01;
***p < 0.001
T: effect of treatment;
H: effect of the time of sampling,
H × T: interaction o the time of sampling and the treatment,
C: effect of the horse

It is important to note that the statistical analysis revealed an interaction of the significant factors “time of collection” and “horse” (p=0.024). We can deduce from this that the individual effect has masked the effect of the treatment.

Although the effect of the treatment was masked by the interaction of the factors “effect of the time of collection” and “effect of the horse”, we can clearly discern a trend for the product THS-MS01 to smooth out changes in the glycaemia, in particular to reduce the initial glycaemia peak to T_{+1.5} and to keep it lower in the long term (in particular at T₊₆). This maintenance of glycaemia at a lower level, while limiting the fluctuations in glycaemia, provides evidence of the diffusion of the animal's energy intake in a regular manner and prolonged over time.

2.2. Faecal Microflora and Activity

2.2.1. Fermentation Products and Parameters

TABLE 6
Effect of the products TSH-MS01, 02 and 03 on the faecal concentration of
total volatile fatty acids, the molar proportions of acetate (C2), propionate
(C3), butyrate (C4) and valerate (C5), the (C2 + C4):C3 ratio, on the
concentrations of D-lactate and L-lactate and on the L:D ratio and on the
pHs measured in the faeces of the horses (n = 6).

| | | | Treatment | | | |
|---|---|---|---|---|---|---|
| | | | TSH- | TSH- | | |
| | R² | SD | MS01 | MS02 | TSH-MS03 | Effects (1) |
| Total VFAs | 0.87 | 16.6 | 69.7 | 50.3 | 78.6 | T (P = 0.0629) |
| (mmol/L) | | | | | | |
| % C2 | 0.98 | 2.0 | 63.2 | 58.8 | 59.4 | T* (P = 0.020) |
| | | | | | | P × T* (P = 0.020) |
| % C3 | 0.96 | 2.0 | 24.6 | 26.6 | 24.0 | P × T** (P = 0.009) |
| % C4 | 0.96 | 0.7 | 8.1 | 9.0 | 9.0 | |
| % C5 | 0.75 | 1.0 | 0.8 | 1.2 | 2.1 | |
| (C2 + C4)/C3 | 0.98 | 0.2 | 2.9 | 2.9 | 3.1 | P × T** (P = 0.008) |
| pH | 0.74 | 0.2 | 6.7 | 6.7 | 6.8 | |
| D-Lactate | 0.91 | 0.6 | 4.8 | 4.3 | 5.1 | |
| (mmol/L) | | | | | | |
| L-Lactate | 0.85 | 1.6 | 6.5 | 5.4 | 8.0 | T (P = 0.064) |
| (mmol/L) | | | | | | |
| L/D | 0.83 | 0.7 | 1.6 | 1.3 | 1.8 | |

(1) significance level:
*p < 0.05;
**p < 0.01
T: effect of treatment;
P × T: interaction of the period and the treatment

Although the interaction of the factors “treatment” and “period” was significant (p<0.05), it was possible to observe that when the products TSH-MS01 and THS-MS03 were distributed to the horses, the total faecal concentration of VFAs and the faecal concentration of L-Lactate were higher (p<0.05), reflecting a greater activity of the faecal microflora.

These results reflect an increase in the energy available (VFAs and L-lactate) for the animal. Moreover, the increase in L-lactate or in the L-lactate:D-lactate ratio is advantageous as this form of lactate can be more easily assimilated (unlike D-Lactate which accumulates in the blood and causes muscle cramps in the animal).

Moreover, when the products TSH-MS01 and THS-MS03 were distributed to the horses, the molar percentage of acetate was greater in the faeces of the horses (p<0.05), reflecting a higher faecal fibrolytic activity.

More specifically, when the horses received the product TSH-MS01, the percentage of acetate was significantly higher whereas the percentage of propionate was significantly lower. These results reflect a further stimulating effect of the product TSH-MS01 on the fibrolytic activity of the faecal microflora, and therefore an increase in the energy available to the animal. In practice, the product TSH-MS01 is preferentially administered to a monogastric herbivore which will have a low-starch, fibre-rich diet. The product TSH-MS01 allows the animal to derive greater benefit from the plant fibres over a longer period. The product TSH-MS01 is particularly suitable for touring horses and recreation horses.

On the other hand, when the horses have received the product TSH-MS03, it is possible to note an even greater increase in VFA and L-lactate. These results reflect a more stimulating effect of the fermentative activity (increase in VFAs) and of the amylolytic and lacticolytic activity of the faecal microflora (increase in L-lactate). In practice, the product TSH-MS03 is preferentially administered to a monogastric herbivore which has to exert a great effort over a short period. The product TSH-MS03 reproduces the effect of an energy diet which is rich in starch although the ration is rich in fibres. As a result, the product TSH-MS03 allows the animal to derive greater benefit from its feed ration rapidly after absorption of its ration. The product TSH-MS03 is particularly suitable for race horses or sport horses.

TABLE 7
Coefficient of variation (%) of the measurements of VFA, lactate and
pH as a function of the treatment received by the horses (n = 6)

| Coefficient of variation | Treatment | | |
|---|---|---|---|
| (%) | TSH-MS01 | TSH-MS02 | TSH-MS03 |
| Total VFA | 33.3 | 30.0 | 45.7 |
| % C2 | 5.0 | 22.7 | 15.3 |
| % C3 | 8.7 | 37.8 | 30.6 |
| % C4 | 23.3 | 30.1 | 20.1 |
| % C5 | 38.1 | 64.5 | 87.9 |
| (C2 + C4)/C3 | 10.8 | 41.2 | 34.0 |
| pH | 3.6 | 2.2 | 5.8 |
| D-Lactate | 28.8 | 26.5 | 29.0 |
| L-Lactate | 40.2 | 40.9 | 24.9 |
| L/D | 73.9 | 40.0 | 64.7 |

For the VFA measurements, the coefficient of variation was numerically lower when the horses had consumed the product TSH-MS01. The product TSH-MS01 tends to reduce the variations between the individuals. This shows that the product TSH-MS01 has an effect on the energy intake exhibiting high reproducibility and stability.

2.2.2. Microbial Counts of the Indigenous Faecal Flora

TABLE 8
Effect of the products TSH-MS01, 02 and 03 on the microbial count
of the total anaerobic, amylolytic, lacticolytic and cellulolytic flora
measured in the faeces of horses (n6).

| | | | Treatments | | |
|---|---|---|---|---|---|
| Flora | | | TSH- | TSH- | TSH- |
| (log10 cfu/mL) | R² | SD | MS01 | MS02 | MS03 |
| Total anaerobic flora | 0.91 | 0.3 | 7.3 | 7.1 | 7.4 |
| Lacticolytic flora | 0.58 | 0.6 | 6.6 | 6.7 | 7.0 |
| Amylolytic flora | 0.76 | 0.7 | 5.0 | 5.3 | 5.5 |
| Cellulolytic flora | 0.88 | 17.5 | 5.6 | 4.7 | 4.8 |

When the horses had consumed the product TSH-MS01, the faecal concentrations of total anaerobic bacteria were numerically increased, reflecting a higher level of activity of the intestinal microflora. The lacticolytic and amylolytic flora are slightly reduced to the benefit of the cellulolytic flora, reflecting a higher fibrolytic activity.

When the horses have consumed the product TSH-MS03, the faecal concentrations of total anaerobic bacteria were further numerically increased, reflecting an even higher level of activity of the intestinal microflora. The lacticolytic, amylolytic and cellulolytic activities were numerically increased, reflecting a higher level of fibrolytic and amylolytic activity.

These results confirm the results of Table 6.

2.3. Digestibility of the Constituents of the Ration

Effect of the products MS-TSH01, 02 and 03 on the in vivo digestibility of the constituents of the ration, using ADL as internal marker (n=6).

When the products TSH-MS01 and TSH-MS03 were distributed, the digestibility of the fibrous constituents of the ration was increased.

TABLE 9
Coefficient of variation (%) of the measurements of digestibility of
the constituents of the ration as a function of the treatment (n = 6)

| Coefficient of variation | Treatment | | |
|---|---|---|---|
| (%) | TSH-MS02 | TSH-MS02 | TSH-MS03 |
| dDM (%) | 3.7 | 9.0 | 8.0 |
| dOM (%) | 3.0 | 7.8 | 6.8 |
| dNDF (%) | 10.2 | 17.3 | 15.9 |
| dADF (%) | 19.7 | 26.0 | 23.5 |
| dhemicelluloses (%) | 3.9 | 12.7 | 11.8 |
| dcellulose (%) | 16.5 | 22.6 | 20.4 |
| dwalls (%) | 8.5 | 10.3 | 15.7 |

For all of the digestibility measurements, the coefficient of variation was numerically lower when the horses had consumed the products TSH-MS01 and TSH-MS03. We can assume that the products TSH-MS03 and even more the product TSH-MS01 tend to reduce the variations between individuals that exist in digestibility measurements. This reflects the reproducibility and the stability of the effects of the supplementation according to the invention.

### Example 2

In this example, the impact of supplementation with TSH-MS01 on the digestibility of the constituents of the ration before, during and after an endurance test was measured.

Reminder of the Protocol Implemented

Animals: Six purebred Arabian horses (two mares and four geldings) aged from 7 to 13 years, with an average weight and a body condition score (BCS) of 422.5 kg±20.7 and 3.2±0.2 respectively at the start of the experiment were used.

The animals were placed in individual boxes (13.3 m²), on artificial bedding (standard wood shavings, Tierwhol, Retteinmaier, France). The horses were put out in individual grass paddocks daily from 10:00 to 17:00. They had a block of mineral salts which they could access at will and had free access to an automatic drinking trough.

Training: The horses were trained in a similar manner. The training consisted of rides (2½ hours), out of doors over varied terrain, 3 to 4 times a week. The trainings were carried out at three gaits, over a distance of approximately 20 km and at an average speed of 10 km/h. The horses were also walked out daily (1 hour at 7 km/h). Specific on-track gallop sessions took place 2 weeks before a test.

Competitions: During the experimentation, the horses took part in two official competitions of 130 km (CEI**) during 2009.

Feeding: Throughout the experimentation period, the horses received a ration based on natural meadow hay and concentrated feed (DP Puissance granules). The daily ration was composed of two feeds of hay, distributed at 10:00 and 16:00, and two feeds of concentrated feed, distributed at 08:00 and 17:30. Outside the experimental periods, the quantities of concentrated feed distributed were estimated as a function of the changes in the horses' weight and body condition in order to maintain their on-form weight. During the measurement periods, the same feeds were distributed, and the quantities fixed according to a fodder:concentrate ratio of 85:15 and an ingestion level of 2.6 kg of hay per 100 kg of body weight.

On the day of the endurance tests, the same feeds were distributed but the distribution timings and the quantities were different: the concentrate was distributed in several small feeds at each vet gate and hay distributed at will the previous evening and on the evening of the race for example.

Supplementation with THS-MS01 was carried out during the three weeks preceding the race and up to 13 days after, according to the experimental protocol described subsequently. The doses of the product THS-MS01 (2 g sachets containing 1. 10¹⁰ CFU) were previously mixed with 50 g soya oil cake and maize flour before being distributed to the animals with the morning feed of granules. During the endurance test, a dose of product was distributed with each feed of concentrate.

Experimental method: The six horses were divided into two groups. The first group (Belik, Riminita and Zaaf) received supplements during the first training period preceding the first race (P1), the other group (Nafar, Kebar and Naya) constituting the control group and the groups were swapped over during the second period (P2), each horse thus being its own control.

The apparent total digestibility measurements were carried out before, during and after two endurance events, corresponding to the two experimental periods P1 and P2. Each period started 7 days before the race and ended 13 days after, making a total duration of 21 days.

Results

Competition Results

During the first period, five of the six horses took part in a 130 km endurance event. During the second period, the six horses took part in a 130 km event.

Feeding

The horses consumed the doses of product without problems throughout the experimental periods, including the day of the race.

Apparent Total Digestibility

The apparent total digestibility is a measurement which allows overall evaluation of the efficiency of the different digestive processes over the entire digestive tract. It represents the percentage in a feed of one of its constituents having disappeared during its passage through the digestive tract, by comparison of the ingested and excreted matter. In our test, the digestibility measurements were carried out using a partial collection of the faeces over 4 consecutive days (Goachet et al., 2009). Thus,
T0: pool 1 is composed of the faeces collected from D−7 to D−4 (before the race),
T7: pool 7 is composed of the faeces collected from D−1 to D+2,
T8: pool 8 is composed of the faeces collected from D0 to D+3,
T9: pool 9 is composed of the faeces collected from D+1 to D+4,
T18: pool 18 is composed of the faeces collected from D+10 to D+13.

The digestibility values obtained in this test are in accordance with the bibliography for the same type of diet (Pagan et al., 1998; Goachet et al., 2009).

In general, the digestibility of the OM and the parietal constituents was higher when the horses had received the TSH treatment (Table 10).

TABLE 10
Average* digestibility and standard deviation (%) of the OM and
the parietal constituents, with or without supplementation.

| (n = 6) | Control | TSH | P value |
|---|---|---|---|
| dOM | 52.9 ± 3.7 | 56.3 ± 2.7 | 0.001 |
| dNDF | 42.4 ± 5.0 | 46.3 ± 3.9 | 0.003 |
| dADF | 37.4 ± 5.1 | 41.0 ± 3.3 | 0.001 |
| d(NDF-ADF) | 48.0 ± 5.3 | 52.3 ± 4.9 | 0.008 |
| d(ADF-ADL) | 42.8 ± 5.8 | 47.0 ± 3.8 | 0.001 |

*Averages of pools 1, 7 and 18.

On the other hand, the coefficients of digestibility of the OM, the NDF and the ADF of pool 7 were significantly higher with the TSH treatment (Table 11).

Pool 7 corresponds to the period around the race: outward transport/race/return transport and 1 day of rest.

Table 11 shows that the animals to which at least one strain of bacterium according to the invention is administered exhibit improved digestibility and assimilability of fibres and cereals compared with the control, even under conditions of physical effort (endurance) and stress (Pool 7).

TABLE 11
Average digestibility (%) of the OM and parietal constituents in
the different pools, with or without supplementation

| | | Pool 1 | Pool 7 | Pool 8 | Pool 9 | Pool 18 |
|---|---|---|---|---|---|---|
| dOM | Control | 53.5 | 51.7 a | 51.6 | 51.8 | 53.4 |
| | TSH | 55.7 | 56.6 b | 55.5 | 55.2 | 56.5 |
| | | | (p ≦ 0.02) | | | |
| dNDF | Control | 42.7 | 40.9 | 40.8 | 41.5 | 43.5 |
| | TSH | 45.6 | 45.8 | 44.5 | 44.7 | 47.5 |
| | | | (p ≦ 0.06) | | | |
| dADF | Control | 38.0 | 36.3 a | 36.5 | 37.6 | 37.9 |
| | TSH | 41.2 | 39.8 b | 39.4 | 39.5 | 42.1 |
| | | | (p ≦ 0.03) | | | |
| d(NDF − | Control | 48.1 | 46.0 | 45.7 | 46.0 | 49.8 |
| ADF) | TSH | 50.7 | 52.4 | 50.2 | 50.6 | 53.6 |
| d(ADF − | Control | 43.5 | 41.6 | 41.8 | 43.0 | 43.3 |
| ADL) | TSH | 47.0 | 45.7 | 45.3 | 45.3 | 48.2 |

1. A method for improving the digestibility and assimilability of fibres and/or cereals in a monogastric herbivorous animal comprising the step of administering to said monogastric herbivorous animal an effective amount of at least one strain of bacterium selected from the group consisting of strains of bacteria of the genera Lactobacillus, Lactococcus, Propionibacterium, Bifidobacterium and Bacillus.

2. The method of claim 1, wherein said step of administering to said monogastric herbivorous animal an effective amount of at least one strain of bacterium selected from the group consisting of strains of bacteria of the genera Lactobacillus, Lactococcus, Propionibacterium, Bifidobacterium and Bacillus consists of administering an effective amount of a mixture of at least two strains of bacteria selected from the group consisting of strains of bacteria of the genera Lactobacillus, Lactococcus, Propionibacterium, Bifidobacterium and Bacillus.

3. The method of claim 2, wherein said mixture of strains of bacteria comprises at least one strain of bacterium of the genus Lactobacillus.

4. The method of claim 1, wherein said strains of bacteria are selected from strains of bacteria of the species L. paracasei, L. casei, L. acidophilus, L. buchnerii, L. farciminis, L. rhamnosus, L. reuteri, L. fermentum, L. brevis, L. lactis and L. plantarum; Lactococcus cremoris and Lactococcus lactis; Propionibacterium jensenii, Propionibacterium acidipropionici, Propionibacterium freudenreichii and Propionibacterium freudenreichii ssp shermanii; Bifidobacterium longum, Bifidobacterium lactis and Bifidobacterium animalis; Bacillus licheniformis, Bacillus subtilis and Bacillus cereus.

5. The method of claim 2, wherein said strains of bacteria are selected from strains of bacteria of the species L. paracasei, L. casei, L. acidophilus, L. buchnerii, L. farciminis, L. rhamnosus, L. reuteri, L. fermentum, L. brevis, L. lactis and L. plantarum; Lactococcus cremoris and Lactococcus lactis; Propionibacterium jensenii, Propionibacterium acidipropionici, Propionibacterium freudenreichii and Propionibacterium freudenreichii ssp shermanii; Bifidobacterium longum, Bifidobacterium lactis and Bifidobacterium animalis; Bacillus licheniformis, Bacillus subtilis and Bacillus cereus.

6. The method of claim 2, wherein said mixture of at least two strains of bacteria is a mixture of at least one strain of L. plantarum and at least one strain of Propionibacterium jensenii.

7. The method of claim 2, wherein said mixture is a mixture of L. plantarum Lp115 and Propionibacterium jensenii P63.

8. The method of claim 1, wherein the strains of bacteria are inactivated.

9. The method of claim 2, wherein the strains of bacteria are inactivated.

10. The method of claim 1, wherein said effective amount of at least one strain of bacterium is administered to said monogastric herbivorous animal by supplementing food intended for said animal with said effective amount of at least one strain of bacterium.

11. The method of claim 2, wherein said effective amount of at least one strain of bacterium is administered to said monogastric herbivorous animal by supplementing food intended for said animal with said effective amount of at least one strain of bacterium.

12. The method of claim 1, wherein said monogastric herbivorous animal is selected from the members of the Equidae and Suidae.

13. The method of claim 2, wherein said monogastric herbivorous animal is selected from the members of the Equidae and Suidae.

14. The method of claim 1, wherein said monogastric herbivorous animal is a member of the horse family.

15. The method of claim 2, wherein said monogastric herbivorous animal is a member of the horse family.

16. The method of claim 1, wherein said monogastric herbivorous animal is a horse or a pony.

17. The method of claim 2, wherein said monogastric herbivorous animal is a horse or a pony.

18. A feed supplement for a monogastric herbivorous animal comprising at least one strain of bacterium selected from the group consisting of strains of bacteria of the genera Lactobacillus, Lactococcus, Propionibacterium, Bifidobacterium and Bacillus.

19. The feed supplement of claim 18, comprising at least one strain of bacterium selected from the group consisting of the species L. paracasei, L. casei, L. acidophilus, L. buchnerii, L. farciminis, L. rhamnosus, L. reuteri, L. fermentum, L. brevis, L. lactis and L. plantarum; Lactococcus cremoris and Lactococcus lactis; Propionibacterium jensenii, Propionibacterium acidipropionici, Propionibacterium freudenreichii and Propionibacterium freudenreichii ssp shermanii; Bifidobacterium longum, Bifidobacterium lactis and Bifidobacterium animalis; Bacillus licheniformis, Bacillus subtilis and Bacillus cereus.

20. The feed supplement of claim 18, comprising at least one strain of bacterium selected from L. plantarum Lp115 and Propionibacterium jensenii P63.

21. A feed for a monogastric herbivorous animal, wherein said feed is supplemented with a feed supplement according to claim 18.

22. A method for regulating glycaemia in a monogastric herbivorous animal, said method comprising the step of administering to said animal a feed supplement according to claim 18.

23. A method for reducing digestive disturbances induced by intense physical exercise and/or stress in a monogastric herbivorous animal, said method comprising the step of administering to said animal at least one strain of bacterium selected from the group consisting of strains of bacteria of the genera Lactobacillus, Lactococcus, Propionibacterium, Bifidobacterium and Bacillus.
